(19) United States
(12) Patent Application Publication
(10) Pub. No.: US 2009/0124545 A1
(43) Pub. Date: May 14, 2009

(54) NOVEL NUCLEIC ACID ENCODING A NOVEL PROTEIN OF CANDIDA ALBICANS AND USES THEREOF

(75) Inventor: Parag Sadhale, Karnataka State (IN)

Correspondence Address: Intellectual Property Dept.;Dewitt Ross & Stevens SC, 2 East Mifflin Street, Suite 600, Madison, WI, 53703-2865, US

(73) Assignee: INDIAN INSTITUTE OF SCIENCE

(21) Appl. No.: 11/997,622

(22) PCT Filed: Aug. 8, 2006
(86) PCT No.: PCT/IN06/00285

§ 371 Date: Feb. 1, 2008

(30) Foreign Application Priority Data
Aug. 8, 2005 (IN) 1088/CHE/2005

(51) Int. Cl. A61K 38/16; C07K 14/00; C07H 21/00; C12N 15/63; A61P 31/10; C12N 1/00; C12P 19/34

(52) U.S. Cl. 514/12; 530/350; 536/23.74; 435/320.1; 435/243; 435/91.1

(57) ABSTRACT

A novel Candida albicans nucleotide and polypeptide, CaSRF1, involved in regulating the morphogenetic transformation and virulence in response to engulfment by the immune response cells of its model host is described. The gene is unique in its ability to affect the virulence-associated morphogenesis in vivo but is not required for the morphogenesis in vitro. The putative membrane localization and its effect on cell wall integrity indicates that it is an ideal anti-candida drug target by virtue of its predicted easy accessibility to lead molecules/chemicals and its ability to affect virulence.

The invention relates to a novel nucleic acid sequence encoding a protein that is specifically expressed in Candida albicans and uses thereof.

### FIELD OF THE INVENTION

The present invention relates to a novel nucleic acid and its protein sequence. Specifically the invention describes a novel gene expressed in Candida albicans that could be used as an anti Candida drug target.

### BACKGROUND OF THE INVENTION

Candida albicans is opportunistic yeast that lives in the gastrointestinal and genitourinary tract of most humans. In a healthy human body, the population of Candida is kept in check by the immune system and by the normal microflora of the respective niches in the host microorganisms. When the immune system is compromised, as in AIDS patients and in patients undergoing immunosuppressive therapy, Candida albicans can cause mucosal as well as systemic infection or “Candida mycosis”. If left untreated, such systemic infections frequently lead to the death of the patients. Candida albicans is a species of particular interest to medical professionals and scientists because a very large fraction of all cases of Candida mycosis are caused by this species.

Two classes of antifungal drugs are used to fight Candida infections. The fungicidal polyene drugs such as amphotericin B act by disrupting membrane function while the fungistatic azoles, such as fluconazole and ketoconazole, act by inhibiting the ergosterol biosynthetic pathway. Amphotericin B is the most effective antifungal drug, but it is more toxic and is less tolerated by the body than the azoles. As a result, azoles have become the drug treatment of choice for many mucosal fungal infections. At present, the therapy principally available for invasive infections is based on relatively few antifungal antibiotics such as the azole derivatives fluconazole and itraconazole or nystatin, amphotericin B and flucytosine. Most of these compounds have serious side effects like tissue toxicity (See Romani et al., Curr. Opin. Microbiol. 6: 338-343, 2003) A serious need in developing newer antifungal drugs has been felt, especially since rise in conditions like AIDS and use of immune suppressive drugs in various medical conditions which has led to significant increase in incidence of Candida infections. Newer drugs, to novel targets in the pathogen could also address a serious problem of drug resistant strains of Candida albicans reported all over the world. Major efforts have been recently focused on identifying newer and unique potential drug targets.

The present invention provides an isolated polynucleotide sequence coding for a protein that is linked to the morphological transition between the yeast to hyphal state of Candida albicans in vivo as well as its ability to survive engulfment by phagocytic macrophages. Furthermore the invention also provides a novel anti-Candida drug target for treating Candida albicans infection.

### SUMMARY OF THE INVENTION

The invention disclosed herein provides a novel gene designated as CaSRF1 expressed by Candida albicans that is involved in modulating the morphogenetic transformation and virulence upon engulfment by the immune response cells of the host. The use of the gene in developing novel anti-candida drug targets for treating fungal infection is also described.

One aspect of the invention is to provide an isolated polypeptide of CaSRF1 gene comprising an amino acid sequence of SEQ ID NO: 2. The invention may also include naturally occurring allelic variant of the sequence given by SEQ ID NO: 2. Furthermore, the polypeptide variant includes any amino acid specified in SEQ ID NO: 2 that may be changed to provide a conservative substitution.

Another aspect of the invention is to provide a nucleic acid molecule comprising a nucleic acid sequence of SEQ ID NO: 1 encoding a polypeptide comprising an amino acid sequence of SEQ ID NO: 2. The invention also includes the nucleotide sequence of the naturally occurring allelic nucleic acid variant of SEQ ID NO: 1. In addition, any single nucleotide polymorphism of SEQ ID NO: 1 is encompassed by the instant invention. The invention also provides a vector comprising the nucleic acid sequence of SEQ ID NO: 1 and a transformed host cell comprising the said vector.

Yet another aspect of the invention is to provide a method of modifying a nucleic acid sequence of SEQ ID NO: 1 by deletion comprising the steps of:

a. generating two primers each about 93-94 nucleotides long;

b. amplifying two different nutritional marker genes URA3 and ADE2;

c. transforming the PCR products generated with URA3 and ADE2 markers in the WT strain CAI8 and

d. isolating the transformants

The primers for the method described herein comprise 5′ terminal sequence of forward primer corresponding to 70 nucleotides immediately upstream of the ATG of the open reading frame of SEQ ID NO: 1 and remaining corresponds to the pUC (forward) primer sequence and the 5′ terminal sequence of the dis(R) primer corresponding to 70 nucleotide sequence immediately downstream of the termination codon TAA of the open reading frame of SEQ ID NO: 1 and the remaining corresponds to the pUC (reverse) primer sequence.

In another aspect, the invention provides an antifungal drug target including a polypeptide sequence of SEQ ID NO: 2 and a carrier.

The invention also provides a composition for treating Candida albicans infections comprising an anti-candida drug target of polypeptide sequence SEQ ID NO: 2 and a carrier.

### BRIEF DESCRIPTION OF THE DRAWINGS

FIG. 1 illustrates the nucleotide sequence of the open reading frame designated as SEQ ID NO: 1.

FIG. 2 illustrates the deduced amino acid sequence of the open reading frame of SEQ ID NO: 1

FIG. 3 illustrates the expression analysis of CaSRF1 in yeast versus hyphae favoring conditions.

FIG. 4a illustrates the deletion strategy used to generate a homozygous deletion mutant PSC2. FIG. 4b illustrates primer sequences used for amplification of the nutritional markers for disruption of the CaSRF1 alleles FIG. 4c effect of the deletion in vitro on membrane stability.

FIG. 5 illustrates the effect of macrophage engulfment on Casrf1/Casrf1 deletion mutant.

FIG. 6 illustrates the survival curve for mice infected with 10⁷ cells of homozygous deletion mutant PSC2, cph1efg1/cph1efg1, Sc5314 or heterozygous mutant strain PSC1.

### DETAILED DESCRIPTION OF THE INVENTION

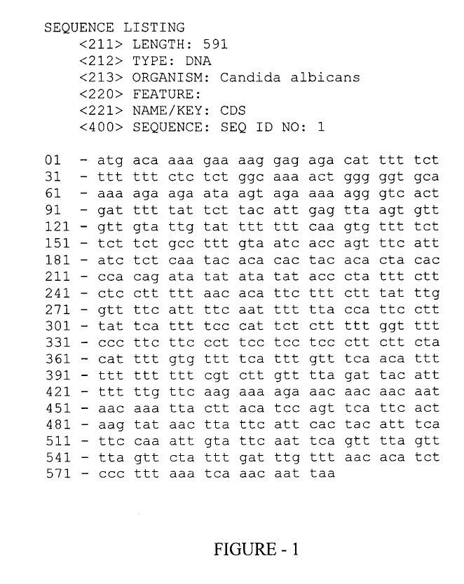

The present invention describes a novel polynucleotide of 591 nucleotides length that encodes a protein that is specifically expressed in the yeast form of Candida albicans in effect plays some role in sensing of altered environmental conditions by the pathogen. The sequence of the polynucleotide given in FIG. 1 and designated as SEQ ID NO: 1 is a part of the gene referred to as CaSRF1/IPF9211.5/CA3142/orf6.5311 /orf19.3713 (http://www.candidagenome.org). Since it was identified as a genetic suppressor of temperature sensitivity of mutant of S. cerevisiae gene RPB4, the gene of the present invention is termed as CaSRF1 (Candida Suppressor of Rpb Four) pending approval from the Candida Genome Database (CGD) curators.

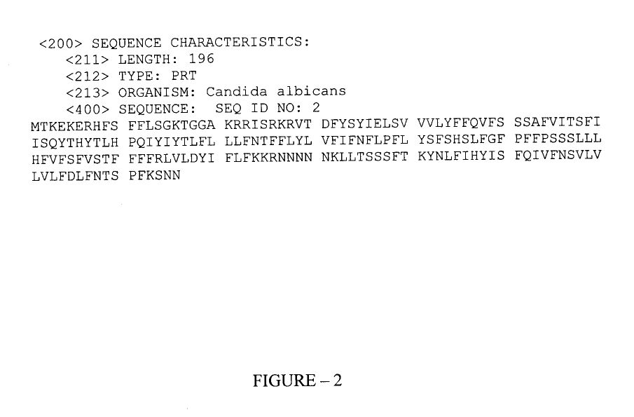

The polynucleotide of the instant invention is capable of encoding a novel polypeptide, which is 196 amino acids in length. The sequence of the polypeptide is given in FIG. 2 and designated as SEQ ID NO: 2. The BLAST analysis (WU-tblastn, V2.0MP-WashU, 13-Dec.-2004) revealed no homologous protein that has significant similarity to the novel protein described herein (SEQ ID NO: 2). The analysis of the sequence using a tool SMART (2) revealed the presence of four trans-membrane domain segments. These segments are hypothesized to be involved in association with cellular membranes.

The expression analysis revealed that the expression of this novel protein represented as SEQ ID NO: 2 is dramatically reduced in cells undergoing hyphal morphogenesis under a variety of conditions such as growth at 37° C. in YPD containing 10% foetal bovine serum or lee's medium or Spider medium (see Annexure I), RPMI containing 10% fetal bovine serum etc (Example 1).

Since Candida albicans is a diploid organism with no known stable haploid state, genetic manipulation necessitates eliminating both the copies of the gene in question. Deletion of this sequence from the genome of Candida albicans eliminates the protein being made in the cell and affects the integrity of the cell walls making the cells sensitive to cell wall disturbing agents such as 0.01% SDS and calcofluor 10 μg /ml present in laboratory culture media (See Examples 2 and 3).

One of the major responses of the C albicans to a variety of environmental conditions is its morphological transition. The transition from the yeast form to the hyphal or pseudohyphal form is tightly associated with the virulence of the organism. The ability of the Candida albicans to form hyphal projections after being engulfed by the phagocytic cells of the immune system contributes greatly to overcoming the cell mediated immunity ensuring its survival in the host and ability to cause infections (Rooney and Klein, 2002, Cell Microbiol 4: 127-137, Gow et al., 2002, Curr. Opin. Microbiol. 5: 366-371)

Candida albicans is capable of differentiating in a reversible fashion between a bud and a hyphal growth form, which is influenced by environmental conditions. For example, pH and temperature influence the transition between bud and hypha while temperature, UV, white blood cell metabolites and so on affect the morphological transition shown by this organism. The morphological changes made by C. albicans in response to environmental cues indicate that the organism uses a sensory mechanism to register and assess environmental alterations. It was observed that the mutant strain lacked the ability to form hyphae piercing the macrophage cells (see Example 4) indicating the role of homozygous deletion mutant PSC2 of the present invention, especially in the macrophages. The inability of the mutant Candida cells to destroy the macrophage cells is seen as an indication of reduced virulence of the mutant cells thus suggestive of the role of this novel protein in macrophages. Furthermore, the protein of the instant invention (SEQ ID NO: 2) appears to be essential for virulence in disseminated candidiasis as seen in mouse model system described in Example 5.

The present invention is described further below by reference to the following illustrative examples.

### EXAMPLE 1

### Expression Analysis And Transcriptional Profile Of CaSRF1

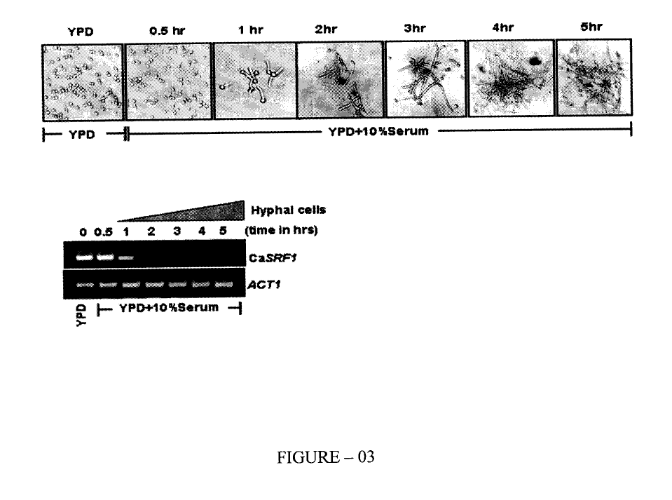

Composition of the media used for culturing the Candida albicans cells is presented in Annexure I. To test whether transcription of CaSRF1 was regulated during hyphal morphogenesis, Northern blots of total RNA of the SC5314 strain incubated in YPD medium favoring the yeast condition and RNAs from cultures showing various extents of hyphae induced by addition of serum (10% v/v) were probed with the DNA fragment spanning the entire open reading frame spanning sequence. The CaSRF1 transcript was detectable at high level in cultures showing a high fraction of cells in the yeast form. As the cells were shifted to the hyphae inducing condition in presence of serum the levels of transcript of this gene were reduced drastically and rapidly being completely shut off by 2 hrs. This was true for many other hyphae inducing growth conditions (FIG. 3). The converse was found to be true in that the cell cultures induced to be mainly (>90% population) in hyphal state when transferred to conditions favoring yeast form the transcript of this gene reappeared although at much slower kinetics as conversion to yeast form takes much longer and only by about 12 hours can one see the culture mainly containing yeast form cells. Both these analyses were carried out using RT PCR technique with open reading frame specific primers.

### EXAMPLE 2

### Deletion of CaSRF1in C. albicans Strain CA18

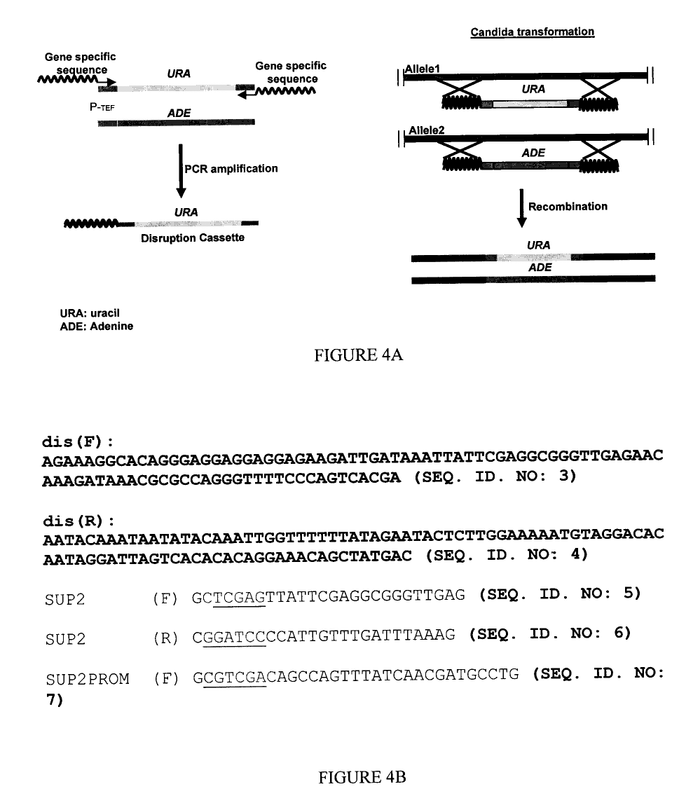

The strategy used for deletion of both the alleles of the CaSRF1 gene is shown in FIG. 4a. In order to generate a CaSRF1 deletion cassette, two primers each about 93-94 nucleotides long (FIG. 4b) were generated. The 5′ terminal sequence of forward primer corresponds to 70 nucleotides immediately upstream of the ATG of the open reading frame and the remaining corresponds to the pUCf primer sequence. The 5′ terminal sequence of the dis(R) primer corresponds to 70 nucleotides immediately downstream of the termination codon TAA of the open reading frame and the remaining corresponds to the pUCr primer sequence. This allowed the amplification of two different nutritional marker genes URA3 and ADE2 respectively cloned previously in the vector pPS5 using PCR amplification method (Ausubel et al, CURRENT PROTOCOLS IN MOLECULAR BIOLOGY, Greene Publishing and Wiley-Interscience, New York, 1995). The PCR products generated respectively with URA3 and ADE2 markers flanked by the homologous sequence to the untranslated regions of the CaSRF1 gene are ˜1.4 and ˜2.5 kb respectively.

These were transformed in the WT strain CA18 C. albicans CAI8 (ade2::hisG/ade2::hisG ura3::imm434/ura3::imm434) (Fonzi and Irwin, 1993, Genetics 143:712-728) by the transformation method employing lithium acetate whereby yeast cells are briefly incubated in buffered lithium acetate and transforming DNA is introduced with carrier DNA. Addition of polyethylene glycol (PEG) and a heat shock trigger DNA uptake (Ausubel et al, CURRENT PROTOCOLS IN MOLECULAR BIOLOGY, Greene Publishing and Wiley-Interscience, New York, 1995). The insertion of the above PCR product in the correct locus in the transformants obtained was confirmed by PCR employing the nutritional marker specific internal primers and a primer upstream of the CaSRF1 gene. The homozygous deletion was confirmed by northern analysis, which showed complete absence of the gene specific transcript as expected.

### EXAMPLE 3

### Functional Characterization Of The Casrf1/Casrf1 Null Mutant Of Strain CAI8

To test whether the Casrf1/Casrf1 i.e. homozygous deletion mutant PSC2 is affected in its ability to show morphological variation like its parent strain, testing was carried out as to how the WT strain CAI8 and the clinical isolate SC5314 behave in presence of serum and some of the other conditions under which C. albicans strains are reported to show hyphal transition associated with virulence. The experiments were carried out at 37° C. In all conditions tested, no significant difference in hyphae formation was observed. Especially the serum induced hyphae formation was seen in the mutant having either no copy of the caSRF1 gene or one copy or two copies as in WT. Similarly the solid media such Lee's medium, YPS medium, YEPD+10% serum as well as media in which pH induced hyphae formation is tested showed no difference. While since the protein is predicted to have four transmembrane domains, it is likely that it plays some role in the membrane/ cell wall integrity. Two chemicals, SDS and calcofluor, resistance to which is dependent on the integrity of the cell wall of the yeast cell (Morenoa et al. FEMS Microbiol. Lett. 226, 159-167) were employed to test if there was any defect in the cells lacking the CaSRF1 protein. It was observed that the homozygous strain was sensitive to 0.05% SDS and 5 μg/ml of Calcofluor.

ADE2 primers referred above are:

Forward primer-CAGATCTCAACACCAATAATTGATGAAAC
Reverse primer-CCTCGAGTAAGAAGGGAAAAGCACCAC

URA3 primers referred above are:

Forward primer
(5′-3′)-CAAGCTT AATAGGAATTGATTTGGATGG
Reverse primer
(5′-3′)-TCTAGAAGGACCACCTTTG

### EXAMPLE 4

### Morphological Changes Of The Homozygous Deletion Mutant PSC2

The Candida albicans strains (Wt. homozygous or heterozygous srf1Δ strain) were co-incubated with mouse macrophage cell line (or peritoneal macrophages) grown in RPMI+10% FCS in 6 well plastic trays for upto 6 hrs and at one hour interval the morphology of the Candida cells was recorded using Leica bright field inverted microscope.

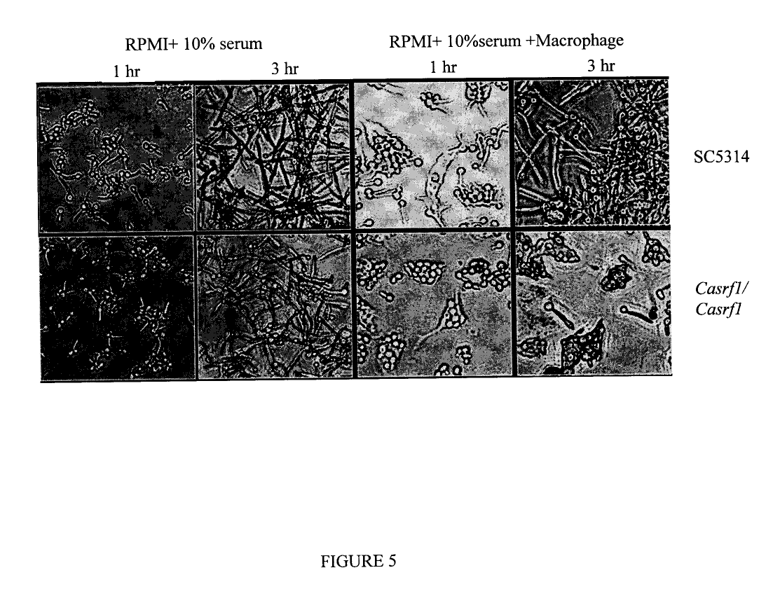

The homozygous deletion mutant PSC2 does not have overall defect in forming hyphae, since the cells incubated in media containing serum as well as other hyphae promoting media (listed in Annexure I) show no difference in the ability of forming hyphae when compared with the clinical isolate SC5314 widely used in laboratory research (FIG. 5). On the other hand the mutant cells were observed to be engulfed by the activated macrophage cells of the immune system but unlike the parent strain were unable to form hyphae piercing the macrophage cells. This inability of the mutant Candida cells to destroy the macrophage cells was seen as an indication of reduced virulence of the mutant cells in turn the observation was considered suggestive of the role of this protein specifically in macrophage.

### EXAMPLE 5

### In Vivo Survival In The Presence Of Homozygous Double Mutant

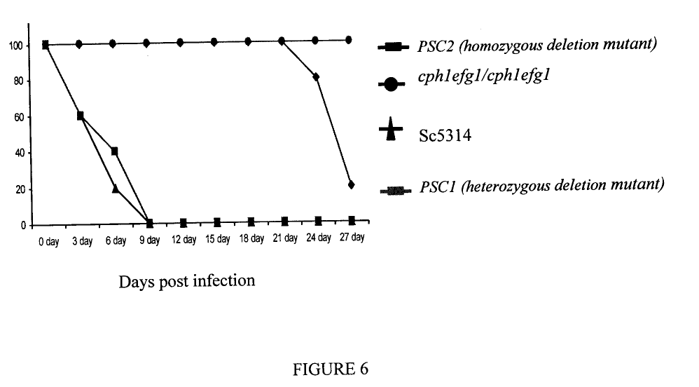

10⁷ cells of the mutant Candida albicans strain per animal were injected in five, 4-week old BALB/c mice via tail vein route. As a control 10⁷ cells of Candida albicans wild type strain SC5314 were injected in five, 4-week old BALB/c mice. The mice in this control group were unable to survive for more than 5 days consistently in three experiments including 5 mice per group in an experiment. The result of a typical experiment is shown in FIG. 6 wherein mice were injected with homozygous deletion mutant PSC2, cph1efg1/cph1efg1, Sc5314 or heterozygous PSC1. The homozygous deletion mutant PSC2 revealed 100% survival similar to the negative control (cph1efg1/cph1efg1) as against the wild type (Sc5314) and the heterozygous mutant (PSC1).

All publications and patent applications referred to in this specification are indicative of the level of skill of those in the art to which the invention pertains.

Other objects, features and advantages of the present invention will become apparent from the foregoing detailed description and examples. It should be understood, however, that the detailed description and the specific examples, while indicating preferred embodiments of the invention, are given only by way of illustration.

### Annexure I

Media compositions of the media used for culturing the Candida albicans cells.

YPD/YPG

| | | |
|---|---|---|
| Yeast Extract | 1 | gm |
| Peptone | 2 | gm |
| Dextrose/Galactose | 2 | gm |

Spider medium

| | | |
|---|---|---|
| Peptone | 1 | gm |
| Yeast Extract | 1 | gm |
| NaCl | 0.5 | gm |
| Mannitol | 1.0 | gm |
| K₂HPO₄ | 0.2 | gm |
| Water | 100 | ml |

Lees Medium

| | | |
|---|---|---|
| (NH₄)₂ SO₄ | 0.5 | gm |
| MgSO₄ | 0.2 | gm |
| K₂HPO₄ | 0.25 | gm |
| NaCl | 0.5 | gm |
| Glucose | 1.25 | gm |
| Biotin | 0.001 | gm |
| DM | 20 | ml |
| Water | 80 | ml |

Synthetic Complete + Serum

| | | |
|---|---|---|
| Dextrose | 2 | gm |
| DM | 20 | ml |
| FCS | 10 | ml |
| Water | 70 | ml |

YPD + Serum

| | | |
|---|---|---|
| Yeast Extract | 1 | gm |
| Peptone | 2 | gm |
| Dextrose | 2 | gm |
| FCS | 10 | ml |
| Water | 90 | ml |

1. An isolated polypeptide comprising an amino acid sequence of SEQ ID NO: 2.

2. The polypeptide of claim 1 that is a naturally occurring allelic variant of the sequence given by SEQ ID NO: 2.

3. The polypeptide of claim 2, wherein the variant is the translation of a single nucleotide polymorphism.

4. The polypeptide of claim 1 that is a variant of SEQ ID NO: 2, wherein any amino acid specified in the chosen sequence is changed to provide a conservative substitution.

5. An isolated nucleic acid molecule comprising a nucleic acid sequence of SEQ ID NO: 1 encoding a polypeptide comprising an amino acid sequence of SEQ ID NO: 2.

6. The nucleic acid molecule of claim 5, wherein the nucleic acid molecule comprises the nucleotide sequence of a naturally occurring allelic nucleic acid variant.

7. The nucleic acid molecule of claim 5, wherein the nucleic acid molecule comprises a single nucleotide polymorphism of SEQ ID NO: 1.

8. The nucleic acid molecule of claim 5, wherein the said nucleic acid molecule comprises a nucleotide sequence of SEQ ID NO: 1.

9. The nucleic acid molecule of claim 5, wherein the said nucleic acid molecule hybridizes under stringent conditions to the nucleotide sequence given by SEQ ID NO: 1 or its complement.

10. A vector comprising a nucleic acid sequence as defined in claim 5.

11. A transformed host cell comprising the vector of claim 10.

12. A method of modifying a nucleic acid sequence of SEQ ID NO: 1 by deletion, the said method comprising the steps of:
a. generating two primers each about 93-94 nucleotides long;
b. amplifying two different nutritional marker genes URA3 and ADE2;
c. transforming the PCR products generated with URA3 and ADE2 markers in the WT strain CA18 and
d. isolating the transformants.

13. The method of claim 12, wherein the said primers comprise 5′ terminal sequence of forward primer corresponding to 70 nucleotides immediately upstream of the ATG of the open reading frame of SEQ ID NO: 1 and remaining corresponds to the pUCf primer sequence and the 5′ terminal sequence of the dis(R) primer corresponding to 70 nucleotide sequence immediately downstream of the termination codon TAA of the open reading frame of SEQ ID NO: 1 and the remaining corresponds to the pUCr primer sequence.

14. An antifungal drug target including a polypeptide sequence of SEQ ID NO: 2 and a carrier.

15. A composition for treating Candida infections comprising an anti-candida drug target of polypeptide sequence SEQ ID NO: 2 and a carrier.


SEQUENCE LISTING (12 sequences)

SEQ ID NO: 1 - DNA, 591 bp, Candida albicans
  CDS (1)..(591)
atgacaaaagaaaaggagagacatttttctttttttctctctggcaaaactgggggtgcaaaaagaagaataagtagaaaaagggtcactgatttttattcttacattgagttaagtgttgttgtattgtatttttttcaagtgttttcttcttctgcctttgtaatcaccagtttcattatctctcaatacacacactacacactacacccacagatatatatatataccctatttcttctcctttttaacacattctttctttatttggttttcattttcaattttttaccattcctttattcattttcccattctctttttggttttcccttcttcccttcctcctcccttcttctacattttgtgttttcatttgtttcaacattttttttttttcgtcttgttttagattacatttttttgttcaagaaaagaaacaacaacaataacaaattacttacatccagttcattcactaagtataacttattcattcactacatttcattccaaattgtattcaattcagttttagttttagttctatttgatttgtttaacacatctccctttaaatcaaacaattaa

SEQ ID NO: 2 - protein, 196 aa, Candida albicans
MTKEKERHFSFFLSGKTGGAKRRISRKRVTDFYSYIELSVVVLYFFQVFSSSAFVITSFIISQYTHYTLHPQIYIYTLFLLLFNTFFLYLVFIFNFLPFLYSFSHSLFGFPFFPSSSLLLHFVFSFVSTFFFFRLVLDYIFLFKKRNNNNNKLLTSSSFTKYNLFIHYISFQIVFNSVLVLVLFDLFNTSPFKSNN

SEQ ID NO: 3 - protein, 196 aa, Candida albicans; identical to SEQ ID NO: 2

SEQ ID NO: 4 - DNA, 93 bp, Artificial Sequence
  PCR primer
agaaaggcacagggaggaggaggagaagattgataaattattcgaggcgggttgagaacaaagataaacgcgccagggttttcccagtcacga

SEQ ID NO: 5 - DNA, 94 bp, Artificial Sequence
  PCR primer
aatacaaataatatacaaattggttttttatagaatactcttggaaaaatgtaggacacaataggattagtcacacacaggaaacagctatgac

SEQ ID NO: 6 - DNA, 26 bp, Artificial Sequence
  PCR primer
gctcgagttattcgaggcgggttgag

SEQ ID NO: 7 - DNA, 25 bp, Artificial Sequence
  PCR primer
cggatccccattgtttgatttaaag

SEQ ID NO: 8 - DNA, 31 bp, Artificial Sequence
  PCR primer
gcgtcgacagccagtttatcaacgatgcctg

SEQ ID NO: 9 - DNA, 29 bp, Artificial Sequence
  PCR primer
cagatctcaacaccaataattgatgaaac

SEQ ID NO: 10 - DNA, 26 bp, Artificial Sequence
  PCR primer
cctcgagtaagaagggaaaagcacca

SEQ ID NO: 11 - DNA, 28 bp, Artificial Sequence
  PCR primer
caagcttaataggaattgatttggatgg

SEQ ID NO: 12 - DNA, 19 bp, Artificial Sequence
  PCR primer
tctagaaggaccacctttg